(19) United States
(12) Patent Application Publication
(10) Pub. No.: US 2013/0174345 A1
(43) Pub. Date: Jul. 11, 2013

(54) OCCUPANT MONITORING SYSTEM

(71) Applicants: Brian LEU, San Jose, CA (US); David CHOW, San Jose, CA (US)

(72) Inventors: Brian LEU, San Jose, CA (US); David CHOW, San Jose, CA (US)

(73) Assignee: MyWellnessGuard Inc., San Jose, CA (US)

(21) Appl. No.: 13/726,892

(22) Filed: Dec. 26, 2012

Related U.S. Application Data
(60) Provisional application No. 61/583,587, filed on Jan. 5, 2012.

(51) Int. Cl. A47C 21/00; G05B 11/01; G06F 15/00; G01P 21/00; A61B 5/11; G01P 15/00

(52) U.S. Cl. CPC A47C 21/00; A61B 5/11; G01P 15/00; G06F 15/00; G01P 21/00; G05B 11/01
USPC 5/694; 600/595; 702/141; 702/104; 700/11

(57) ABSTRACT

Example implementations are directed to a system that can be used to monitor the state of an occupant of a structure, such as a bed or a mattress. The states that can be monitored include whether or not the occupant is present, the position of the occupant, entry or exit of the occupant, and other signs that can be detected via movement, such as activity level, breathing, epileptic seizures, and heart rate. Example implementations involve one or more accelerometers disposed on the structure, such that the movement or changes in position by the occupant is transferred to the accelerometers, and a computing system to process the data from the accelerometers.

### CROSS REFERENCE TO RELATED APPLICATION

This U.S. patent application is based on and claims the benefit of domestic priority under 35 U.S.C 119(e) from provisional U.S. patent application No. 61/583587, filed on Jan. 5, 2012, the entire disclosure of which is incorporated by reference herein in its entirety.

### BACKGROUND

1. Technical Field

Systems, methods and example implementations described herein are generally directed to monitoring of occupants, and more specifically, to bed occupancy monitoring and patient vital sign monitoring.

2. Related Art

Systems in the related art include bed occupancy monitors, which alert caregivers when a patient is on or off a bed. Related art systems may also involve a movement monitor which tracks patient movement to ensure the patient has been moved enough to prevent bed sores. Related art systems may also involve patient vital sign monitors.

Related art occupant monitoring systems use various sensing mechanisms such as pressure sensors, weight sensors, air-pressure sensors, and capacitive sensors, to implement the monitoring functions. Each of these sensing mechanisms has capabilities and limitations.

### SUMMARY

Aspects of the present application may include a system, which may involve a sensor sheet having one or more accelerometers configured to detect one or more surface deflections of an occupant supporting structure and a module configured to record data based on the one or more detected surface deflections and to communicatively connect with a computing device.

Aspects of the present application may further include a computer readable storage medium storing instructions for executing a process. The instructions may include processing data received from a sensor sheet having one or more accelerometers configured to detect one or more surface deflections of an occupant supporting structure.

Aspects of the present application may further include a sensor sheet, which may involve one or more accelerometers configured to detect one or more surface deflections of a mattress; and one or more fasteners configured to fasten the sensor sheet to the mattress.

### BRIEF DESCRIPTION OF THE DRAWINGS

FIG. 1 illustrates an example implementation of an occupant monitoring system.

FIG. 2 illustrates an accelerometer attachment, in accordance with an example implementation.

FIG. 3 illustrates multiple occupant monitor systems managed by a computing device, in accordance with an example implementation.

FIG. 4 illustrates a flow diagram for determining occupant states, in accordance with an example implementation.

FIG. 5 illustrates a flow diagram for determining vital signs from sensor measurements, in accordance with an example implementation.

FIG. 6 illustrates an example implementation of an accelerometer integrated circuit.

### DETAILED DESCRIPTION

Example implementations of the present application relate to monitoring a subject in a supporting structure (e.g., an occupant supporting structure such as a bed or a mattress), without the need for the subject's direct attachment with the monitoring sensor, thereby allowing the subject to freely move without having to consider physical connection to a device.

Example implementations involve the use of one or more accelerometers to accomplish the monitoring functions. In contrast to the related art, example implementations involving accelerometers can be made relatively unnoticeable to the occupant. The accelerometers can be made to be sensitive to the surface deflections of the bed, and thereby sense the occupant's position and motion, without having to be in contact with or directly adjacent to (e.g., underneath) the occupant.

Example implementations therefore involve a system to monitor the state of an occupant in an occupant supporting structure such as a bed or a mattress. The system may include one or more accelerometers disposed on the structure, along with a computing device to process the data from the accelerometers. An accelerometer is a sensing device which can produce an output related to the acceleration that the accelerometer experiences. Gravity is experienced as an acceleration force, so the accelerometer's output changes as its orientation relative to the direction of gravity changes. By attaching an accelerometer on or near the surface of the bed, small changes in the tilt or deflections in areas of the bed surface can be detected.

In an example implementation of the system, accelerometers are attached at fixed locations on a flexible sensor sheet. The sheet can be fastened to a support structure such as a bed in the manner of a cover such as a mattress cover, with one or more fasteners. As the occupant enters or exits the support structure (e.g., bed in this implementation, but not limited thereto), the surface of the bed with the flexible sensor sheet bends or unbends in response. The bending/unbending can involve having the sensor sheet being at an initial or default position, wherein the sensor sheet flexes or otherwise changes from the initial or default position to another position, and wherein the sensor sheet may eventually return back to the initial or default position. Thus, while the occupant is on the bed, even slight motions and changes in position of the occupant result in measurable changes to the accelerometer outputs. Those output data are collected by a computing device and the state of the occupant is determined from the data.

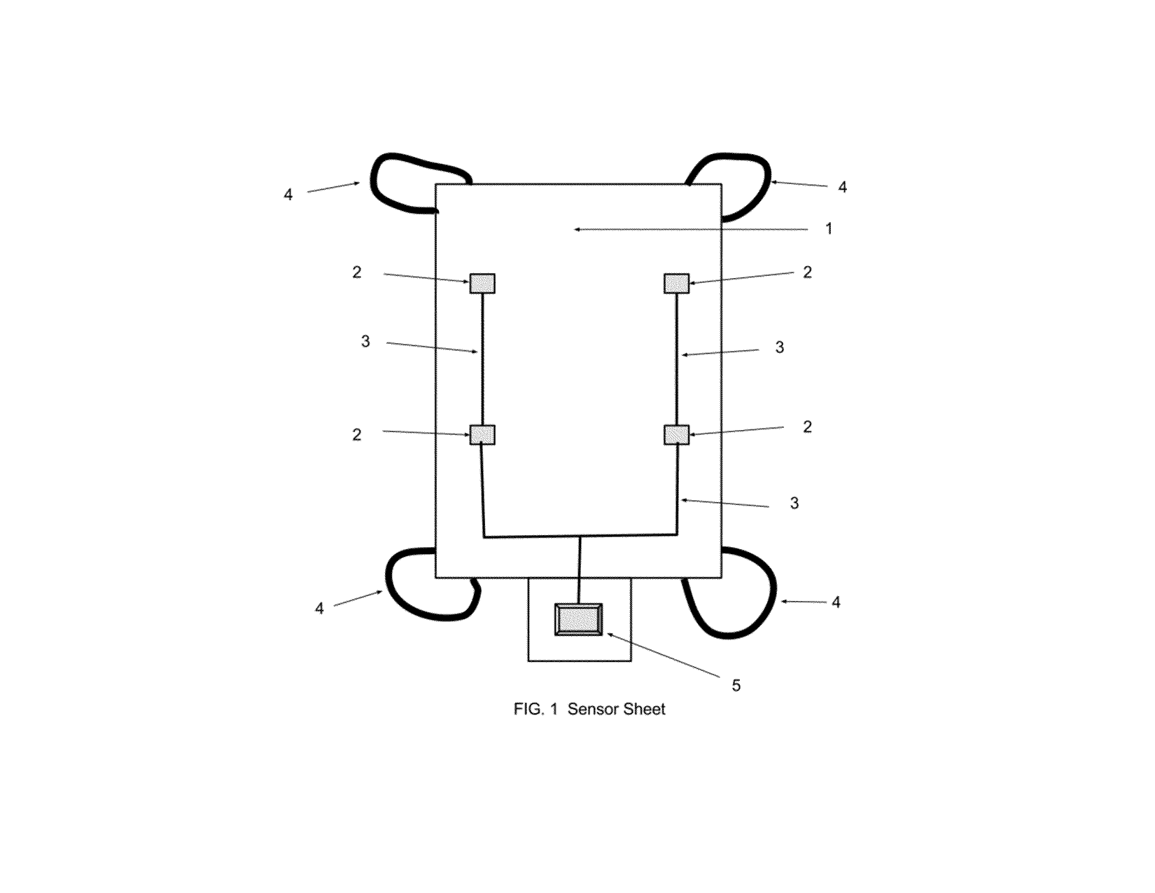

FIG. 1 illustrates an example implementation of an occupant monitoring system. In this example implementation, a sensor sheet 1 utilizes four accelerometers 2 attached along the periphery of a flexible sheet. The number and placement of accelerometers can be varied depending on the desired spatial resolution and coverage area. The accelerometers are connected to a module 5 through sets of wires 3. Each wire set 3 contains wire connections for power and data communications, however, other configurations may also be used depending on the desired implementation (e.g., have the accelerometers connect to the module wirelessly with each accelerometer having its own power source, etc.)

The module 5 provides power to the accelerometers 2, as well as a computer readable medium to collect, process, and relay data. The module itself may be powered by one or more batteries, or plugged with an AC/DC adapter to a main power outlet if desired. The computer readable medium includes tangible media, such as flash memory, random access memory (RAM), hard disk drives (HDD), and so forth. Alternatively, a computer readable signal medium can be utilized, which includes signal media such as carrier waves.

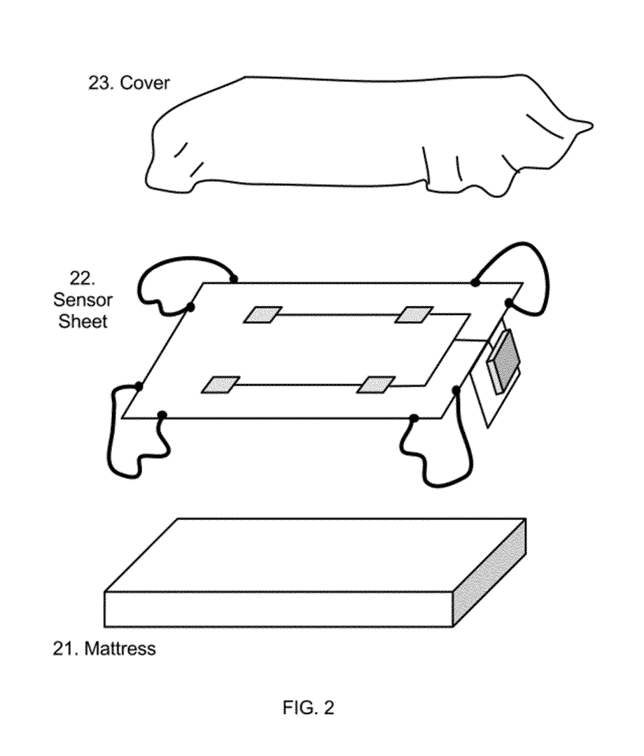

FIG. 2 illustrates an accelerometer attachment, in accordance with an example implementation. Specifically, FIG. 2 illustrates an example implementation the sensor sheet being disposed on an occupant supporting structure, with one or more fasteners configured to fasten the sensor sheet to the occupant supporting structure. In this example implementation, the occupant supporting structure is a mattress. The sensor sheet 22 is fastened to a bed mattress 21 by fasteners in the form of corner straps disposed on the corners of the sensor sheet, each of which configured to be placed around a corner of the mattress. The straps in this example implementation are elastic and adjustable so that they can provide a snug fit to the mattress. However, other fastening structures known to one of ordinary skill in the art may also be used to fasten the sensor sheet to the mattress, depending on the desired implementation. A mattress cover 23 can be used to cover the sensor sheet to make washing more convenient, depending on the desired implementation. Mattress covers or bed sheets do not substantially interfere with the sensors.

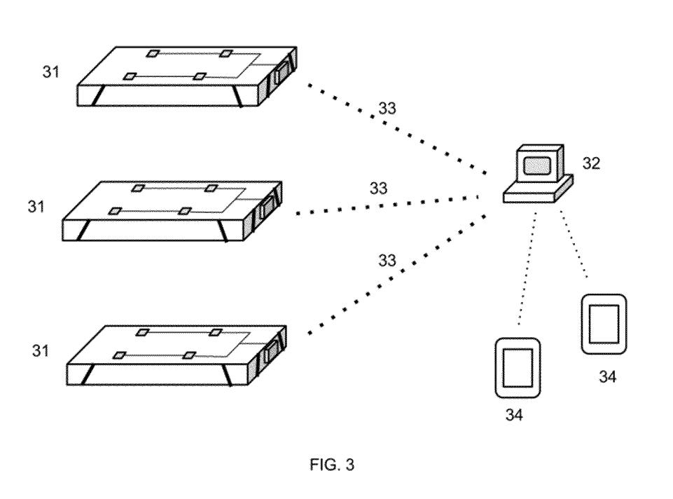

FIG. 3 illustrates an example of a multitude of systems sharing a computing device database, in accordance with an example implementation. The mattresses 31 are shown with the sensor sheets. Each sheet contains a module to conduct data processing local to the individual mattress, but it can be possible for facilities with multiple mattresses to collect information from each mattress to a central location. The module on each mattress can be connected to a computing device 32 via a data connection 33, which could be implemented with wires or in wireless manner.

The computing device 32 acts as a server which can issue notifications to remote devices 34, such as a tablet or smartphone, via a wireless connection such as a Wi-Fi or 3G network. Caregivers can use the remote devices 34 to view database information about the patients, and also to enter responses to notifications. The computing device 32 can maintain a database and keep track of patients, beds, users, caregivers, notifications, and responses, and can be implemented as a computer readable storage medium or a computer readable signal medium.

Notifications can take various forms depending on level of urgency. For example, a notification may show up as a pop-up interface and output an audio signal on the caregiver's device (e.g., remote device) 34. Levels of urgency may be indicated with different text, colors or sounds. Notifications can include instructions for the caregiver, and require an acknowledgement or particular response. The notifications are triggered by conditions determined in a configurable rule-set. The rule-set takes into account the user profiles. The patient's profile includes configurable threshold and calibration levels to set the conditions for the sensor inputs to trigger notifications.

For example, but not by way of limitation, a patient prone to epileptic seizure can have the profile calibrated such that an alert is issued when the sensor sheet detects movement above a specified threshold level. In another example, a patient prone to fall injury may have a calibrated profile such that a notification is sent to a nurse when movement is detected towards the edge of the bed, or when the patient leaves the bed entirely. A patient's user profile may also contain information involving patient history, preferences, medications, restrictions, and other information such as weight, age, family contacts, doctor, or other medical information. The nursing staff may also have profiles, which could include information such as history, capabilities and availabilities. For example, a nurse's profile can be configured such that the nurse receives notifications only from certain patients.

Software on the computing device 32 can be configured to coordinate responses to the inputs and notifications from the various sources, so that responses and resources can be delegated as desired. The sensor sheets provide inputs, but other sensors, such as blood pressure monitors or oximeters, may also be present providing inputs as well. A set of inputs may also come from the patient manually such as an emergency call button or a remote device that controls the environment such as for turning on/off the room lights.

Inputs may also come from nursing staff carrying remote devices 34 that control aspects of the environment. The configurable rule-set may further include rules for governing which personnel or patients have access to which controls. User profiles can provide user preferences and restrictions. Other inputs can be provided automatically, such as the time of day, depending on the desired implementation. For example, a room with a window may not need the lights on during daylight hours.

The configurable rule-set can further include rules regarding how to respond to the given inputs. Taking the inputs and rules into account the computing device 32 can issue responses depending on resources available. For example, a facility may have different personnel working different shifts, and in different locations. The computing device 32 can be made aware of the availability and location of the personnel who are logged in via their remote devices, and can send an alert or request the closest available nurse to respond and help a patient in an example implementation. When the nurse has resolved the request, the nurse can log the response as resolved via remote device 34. The computing device 32 can then obtain the availability of the nurse for another response. The record of responses and resolutions can provide an audit trail to keep track of caregiver performance and ensure patient care. The computing device 32 can also be used to control (e.g., automatically) non-emergency responses such as adjusting light level, room temperature, or other environmental aspects. For example, the room light may be automatically dimmed at night when the occupant is detected to be in bed for some time, and movement is at low level, indicating the occupant is sleeping.

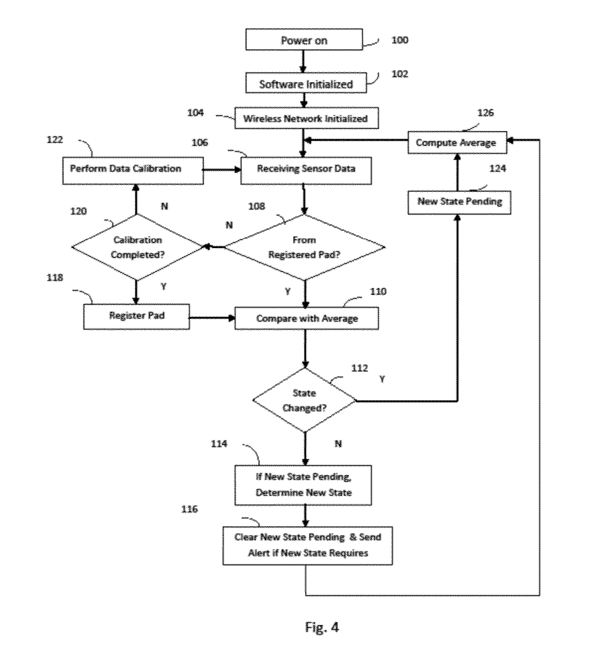

FIG. 4 illustrates the sequence of processing by the computing device to detect occupant state changes in general. At 100, the computing device is powered on. At 102, the software in the device is initialized. At 104, the wireless network is then initialized. When a sensor sheet or pad is powered up, the sensor sheet sends sensor data via wireless network to the computing device at 106, and checks if data received is from a known sensor sheet at 108 (already registered). If the sensor sheet is known, then data is compared with the average data at 110. The ‘average’ may be computed from various methods such as Simple Moving Average (SMA), Cumulative Moving Average, Weighted Moving Average and Exponential Moving Average etc., or other methods known to one of ordinary skill in the art, depending on the desired implementation.

At 112, the computing device then determines if the sensor sheet is in a steady state. If the new data exceeds the average by a threshold (e.g., predefined), the computing device can determine that the sensor sheet is not in steady state and a new state is pending. The new state pending flag can then be set as shown at 124, and the new average is computed at 126. If the new data does not exceed the average and the new state flag is set, the new state is computed at 114. The new state pending flag is cleared and an alert is sent if the new state is configured to send alert at 116. The average is updated with the new data at 126. If data is from an unregistered sensor sheet, the computing device checks if the new sensor sheet is in a calibration state at 120. If calibration has completed for this pad, mark the sensor sheet as registered and ready for data processing. If the sensor sheet is still in calibration, record the data and perform calibration at 122.

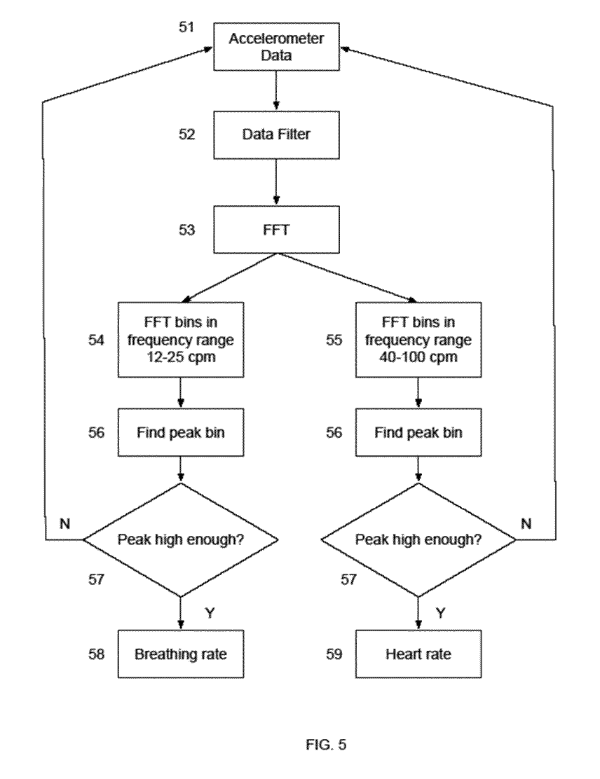

FIG. 5 illustrates a flow diagram for determining vital signs from sensor measurements, in accordance with an example implementation. In example implementations, vital signs can be extracted from the accelerometer measurements, without the need for other additional sensors. FIG. 5 illustrates an example implementation of determining the breathing rate and heart rate from accelerometer data, which can be implemented in a computer readable storage medium at either the sensor sheet 22 or the computing device 32. At 51, the accelerometer data is recorded over time while the occupant is present. At 52, the data is filtered for spurious data points, for example by truncating values outside of expected range. At 53, a Fourier Transform calculation is performed on the filtered data to convert the data from the time domain to the frequency domain. Algorithms such as Fast Fourier Transforms (FFT) can be utilized to perform the Fourier calculation, however, other implementations known to one of ordinary skill in the art can also be used to perform Fourier Transforms. In the example implementation of FIG. 5, the output of the FFT includes values related to the amount of energy the input signal has at a particular frequency. Ranges of frequencies are grouped into bins, and applicable ranges can then be analyzed. In the example implementation of FIG. 5, the implementation assumes that an adult person is expected to breathe between 12 to 25 cycles per minute (cpm), and have a heart beat between 40 to 100 cpm. Therefore at 56, a peak bin value is found within the ranges for breathing (shown at 54) and heart rate (illustrated at 55) respectively. At 57, the peak value is tested 57 to determine if the peak is high enough or meets a threshold compared to average and edge bins to be significant enough for a valid reading. If the peak is determined to be high enough or meets a certain threshold, further output may be determined and produced, such as the breathing rate (shown at 58) and/or heart rate (shown at 59) respectively. If the reading is not valid, then a new set of data can be obtained. Additional bin configurations can be used depending on the desired implementation and the desired vital sign to be determined in a similar manner as described above.

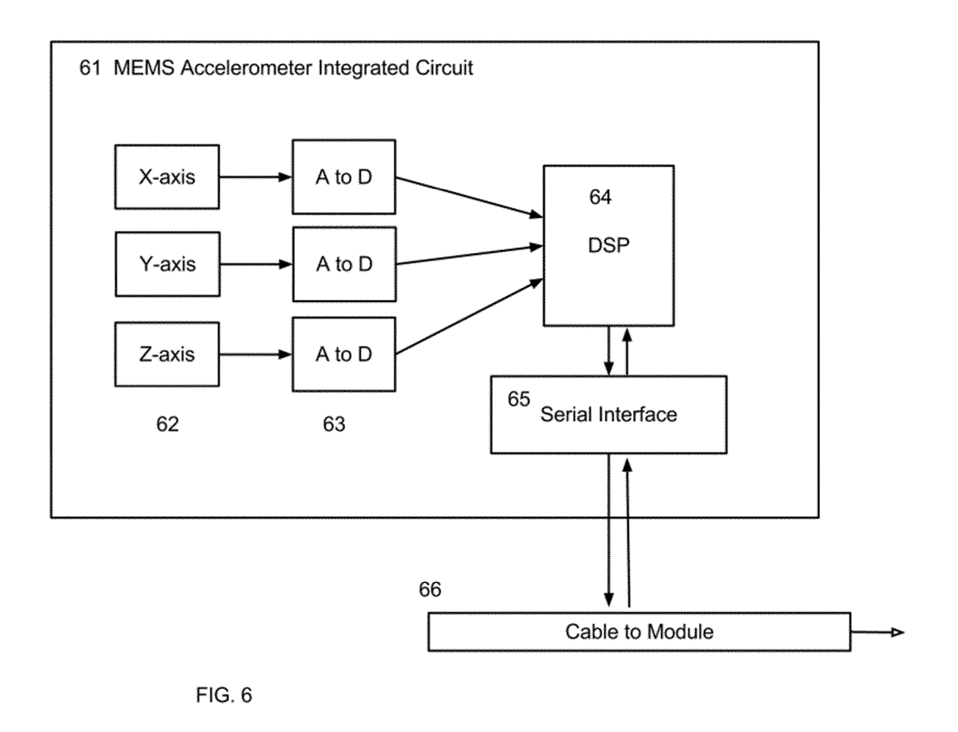

FIG. 6 illustrates an example implementation of an accelerometer integrated circuit. The accelerometer is implemented with MEMS (Micro Electro-Mechanical System) technology. MEMS devices can be manufactured using the same steps as processing silicon integrated circuits (IC's), so functions that can be implemented by silicon IC's can be integrated with the accelerometers onto a single chip 61. A desirable configuration is to have three accelerometers 62 corresponding to the three orthogonal spatial axes. Integrated on the chip are Analog-to-Digital converters 63 to convert the analog signals from the accelerometers to digital format. On-chip digital signal processing (DSP) 64 performs digital functions such as filtering of data. A serial interface 65 provides a means to communicate to an external processing module via cable 66. The cable 66 is a bundle of serial interface wires and power wires. The cable is connected to the external module 5 (FIG. 1) which communicates with, controls, and powers the accelerometer circuitry. Further, other accelerometer configurations may be utilized for implementing a desired implementation of the present application, including, but not limited to, capacitive, optical, resonance, strain gauge, and so on, as well as providing for wireless communication between accelerometers and the processing module, if desired.

Furthermore, some portions of the detailed description are presented in terms of algorithms and symbolic representations of operations within a computer. These algorithmic descriptions and symbolic representations are the means used by those skilled in the data processing arts to most effectively convey the essence of their innovations to others skilled in the art. An algorithm is a series of defined steps leading to a desired end state or result. In the example implementations, the steps carried out require physical manipulations of tangible quantities for achieving a tangible result.

Moreover, other implementations of the present application will be apparent to those skilled in the art from consideration of the specification and practice of the example implementations disclosed herein. Various aspects and/or components of the described example implementations may be used singly or in any combination. It is intended that the specification and examples be considered as examples, with a true scope and spirit of the application being indicated by the following claims.

What is claimed is:

1. A system, comprising:
a sensor sheet comprising one or more accelerometers configured to detect one or more respective surface deflections of an occupant supporting structure, and a module configured to record data based on the one or more detected surface deflections and to communicatively connect with a computing device.

2. The system of claim 1, wherein the computing device is further configured to send a notification to a remote device based on the received data meeting a condition.

3. The system of claim 2, wherein the remote device is configured to connect to the computing device by a wireless network, and to adjust controls associated with the occupant supporting structure by the wireless network.

4. The system of claim 3, wherein the controls associated with the occupant supporting structure comprises environmental controls of a room containing the occupant supporting structure.

5. The system of claim 2, wherein the condition comprises a configurable rule-set based on a profile of the occupant, the configurable rule-set comprising a calibration directed to the occupant.

6. The system of claim 1, wherein the computing device is further configured to determine vital sign data based on the processing of the data from the sensor sheet.

7. The system of claim 1, wherein the sensor sheet further comprises one or more fasteners configured to fasten the sensor sheet to the occupant supporting structure, and wherein the occupant supporting structure comprises a mattress.

8. A computer readable storage medium storing instructions for executing a process, the instructions comprising:
processing data received from a sensor sheet comprising one or more accelerometers configured to detect one or more surface deflections of an occupant supporting structure.

9. The computer readable storage medium of claim 8, wherein the instructions further comprise sending a notification to a remote device based on the processed data meeting a condition.

10. The computer readable storage medium of claim 9, wherein the condition comprises a configurable rule-set based on a profile of the occupant, the configurable rule-set comprising a calibration directed to the occupant.

11. The computer readable storage medium of claim 8, wherein the instructions further comprise adjusting controls associated with the occupant supporting structure based on a received command.

12. The computer readable storage medium of claim 11, wherein the controls associated with the occupant supporting structure comprises environmental controls of a room containing the occupant supporting structure.

13. The computer readable storage medium of claim 8, wherein the instructions further comprise determine vital sign data based on the processing.

14. The computer readable storage medium of claim 8, wherein the occupant supporting structure is a mattress.

15. A sensor sheet, comprising:
one or more accelerometers configured to detect one or more surface deflections of a mattress;
one or more fasteners configured to fasten the sensor sheet to the mattress.

16. The sensor sheet of claim 15, further comprising a module configured to record data based on the one or more detected surface deflections and to communicatively connect with a computing device.

17. The sensor sheet of claim 16, wherein the module is configured to communicatively connect with the computing device by a wireless network.

18. The sensor sheet of claim 15, wherein the one or more fasteners are configured to fasten the sensor sheet to one or more corners of the mattress.

19. The sensor sheet of claim 15, further comprising an adapter configured to connect to a power outlet.

20. The sensor sheet of claim 15, further comprising a power source configured to utilize one or more batteries.
